(19) United States
(12) Patent Application Publication
(10) Pub. No.: US 2002/0031518 A1
(43) Pub. Date: Mar. 14, 2002

(54) Plasminogen fragment having activity to inhibit tumor metastasis and growth and process for preparing same technical field

(75) Inventors: Wataru Morikawa, Kumamoto-shi (JP); Seiji Miyamoto, Kumamoto-ken (JP)

Correspondence Address: BROWDY AND NEIMARK. P.L.L.C., ATTORNEYS AT LAW, SUITE 300, 624 NINTH STREET, N.W., WASHINGTON, DC, 20001-5303, US

(73) Assignee: Juridical Foundation the Chemo-Sero-Therapeutic Research Institute, Kumamoto-shi (JP)

(21) Appl. No.: 09/989,388

(22) Filed: Nov. 21, 2001

Related U.S. Application Data
(63) Continuation of application No. 09/269,720, filed on Apr. 6, 1999.
Application No. 09/269,720 is a 371 of international application No. PCT/JP97/03635, filed on Oct. 9, 1997.

(30) Foreign Application Priority Data
Oct. 9, 1996 (JP) 287651/1996

(51) Int. Cl. A61K 39/395; C12N 9/24

(52) U.S. Cl. 424/174.1; 435/200

(57) ABSTRACT

The present invention provides a plasminogen fragment with the activity to inhibit tumor metastasis and growth, a process for preparing said fragment and an agent for inhibiting tumor metastasis and growth comprising said fragment as an active ingredient. The plasminogen fragment of the present invention is prepared from a elastase lysate of Lys-Plasminogen, which is produced by treating a natural plasminogen with plasmin, and preferably exhibits a strong heparin binding activity. The agent for inhibiting tumor metastasis and growth comprising the plasminogen fragment as an active ingredient of the present invention is useful for clinically treating solid cancers such as lung cancer or colon cancer.

### TECHNICAL FIELD

The present invention relates to a novel plasma protein fragment having a biological activity and to a process for preparing said protein fragment. More particularly, the present invention relates to a plasminogen fragment having an activity to inhibit tumor metastasis and growth, an agent for inhibiting tumor metastasis and growth comprising said plasminogen fragment as an active ingredient and a process for preparing said plasminogen fragment. Said plasminogen fragment is an elastase lysate of Lys-Plasminogen (hereinafter also referred to as “Lys-Plg.”), one of N-terminal modified plasminogens, and exhibits a high heparin binding activity in a preferred embodiment of the present invention. The elastase lysate of Lys-Plg. of the present invention, especially the Lys-Plg. fragment with a high heparin binding, is useful in the field of biochemistry or medicire, for example, for clinical treatment of solid cancers such as lung cancer or colon cancer.

### BACKGROUND ART

Plasminogen, a plasma protein having a molecular weight of 80,000, is a precursor of the enzyme plasmin involved in fibrinolytic system of blood coagulation. Plasminogen is a glycoprotein and about 10 isomers with different isoelectric points are known based on types of glycosylation. Although plasminogen per se is not enzymatically active, it is transformed into active plasmin with an activity to lyse fibrin coagulant by restricted degradation with plasmin, urokinase or plasminogen activator. Plasminogen comprises five Kringle domains (Kringle 1 to Kringle 5) and a serine protease domain with an active center (see FIGS. 1 and 2). Since plasminogen binds to lysine through the property of said Kringle domains, it can specifically be prepared using a lysinebound carrier.

As shown in FIG. 1, as a result of restricted degradation of plasminogen with elastase, three fragments, i.e. a fragment ranging from the amino acid residue 79Tyr to Kringle 3, a fragment of Kringle 4 domain, and a fragment of a serine protease domain including Kringle 5. These three fragments are referred to as “Lysine Binding Site I” (hereinafter also referred to as “LBS-I”), “Lysine Binding Site II” (hereinafter also referred to as “LBS-II”) and “mini-Plasminogen” (hereinafter also referred to as “mini Plg.”), respectively [Davidson J. F. et al., The primary structure of human plasminogen: isolation of two lysine-binding fragments and “mini-”plasminogen (MW: 38,000) by elastase-catalyzed-specific limited proteolysis, Raven Press, New York, vol. 3, 191-209, 1978].

For plasminogen, both Glu-Plasminogen (hereinafter also referred to as “Glu-Plg.”) with Nterminal Glu residue and Lys-Plasminogen (Lys-Plg.) with Nterminal Lys residue are known. The former is an intact plasminogen whereas the latter is produced by cleavage at Lys76-Lys77 at the N-terminal by the action of plasmin when plasminogen is activated (FIG. 2). Lys-Plg. also encompasses those with N-terminal Val or Met in addition to that with N-terminal Lys. When these Lys-Plg. are compared with Glu-Plg., several properties are distinct, for example, Lys-Plg. is much more promptly activated with urokinase than Glu-Plg., or Lys-Plg. has a higher fibrin binding activity than Glu-Plg., and the like. Such different properties deem to result from difference in higher-order structure of both plasminogen molecules, especially difference in conformation of Kringle 1 [Lerch P. G. et al., Localization of individual lysine-binding regions in human plasminogen and investigations on their complex-forming properties, Eur. J. Biochem. 107: 7-13, 1980].

Recently, O'Reilly et al. reported that LBS-I obtained from a lysate of human Glu-Plg. exhibits a vascularization inhibiting activity and inhibits postmetastatic growth of cancers [O'Reilly M. S. et al., Angiostatin: a navel angiogenesis inhibitor that mediates the suppression of metastases by a lewis lung carcinoma, Cell 79: 315-328, 1994]. The present inventors have repeated the experiments as described using plasminogen lysine-binding site I obtained by treating Glu-Plg. with elastase (hereinafter also referred to as “Glu-LBS-I” to discriminate from Lys-Lysine Biding Site (Lys-LBS-I) in accordance with the present invention). However, although Glu-LBS-I inhibited vascularization and metastasis of cancers to some degree, inhibition level was not significant as compared from control.

In view of the above problems, the present inventors have hypothesized that a desired activity can be exhibited only when plasminogen is treated with certain substance (enzyme) prior to treatment with elastase and cannot be obtained when plasminogen is directly treated with elastase to give Glu-LBS-I. Based on this hypothesis, plasminogen was first digested with plasmin, prior to elastase degradation, to prepare a molecule with N-terminal Lys residue, i.e. Lys-Plg., which was then subject to elastase degradation to prepare Lys-Lysine Binding Site I (Lys-LBS-I) which is distinct from the Lysine Binding Site I of O'Reilly et al. (Glu-LBS-I) in their N-terminal amino acid residue. Effects of this molecule (Lys-LBS-I) on cancer metastasis and growth was investigated.

As a result, it was surprisingly found that LysLBS-I exhibits a strong activity to inhibit tumor metastasis and growth, said Lys-LBS-I being obtained by treating Lys-Plg., the lysate product of plasminogen with plasmin, with elastase. Nature of this substance with the activity to inhibit tumor metastasis and growth was further investigated. As a result, it was found that Lys-LBS-I comprises fragments capable of binding to a heparin-carrier at a low ionic strength. That is, it was found that the desired activity to inhibit tumor metastasis and growth was exhibited only by Lys-LBS-I, which was prepared by elastase degradation of the Lys Form product, i.e. plasminogen with N-terminal Lys residue, prepared by plasmin degradation of Glu-Plg. Furthermore, it was also found that, by utilizing such heparin-binding property, the fragments with the activity to inhibit vascularization could easily be prepared with a heparin-bound carrier and as such the present invention was completed.

### DISCLOSURE OF INVENTION

The present invention provides a plasminogen fragment having the activity to inhibit tumor metastasis and growth, said plasminogen fragment being an elastase lysate of Lys-Plg. and having a high heparin binding activity in a preferable embodiment.

The present invention also provides a process for preparing said plasminogen fragment having the activity to inhibit tumor metastasis and growth. The process of the present invention comprises the following steps: (1) plasminogen is treated with plasmin etc. to produce LysPlg.; (2) a Lys-Plg. containing solution is treated with elastase to give fractions of the fragment comprising Kringle 1 to Kringle 3 (Lys-LBS-I); and (3) among the obtained fractions, a fraction of the fragment with a strong heparin binding activity is selected to give a desired plasminogen fragment having the activity to inhibit tumor metastasis and growth.

The present invention further provides an agent for inhibiting tumor metastasis and growth comprising as an active ingredient said plasminogen fragment having the activity to inhibit tumor metastasis and growth.

### BRIEF DESCRIPTION OF DRAWINGS

FIG. 1 is a schematic representation showing products obtained by elastase degradation of plasminogen.

FIG. 2 is a schematic representation showing products obtained by restricted degradation of plasminogen with plasmin followed by elastase treatment.

FIG. 3 is a graph showing an elution pattern of Lys-LBS-I in immunoaffinity chromatography using heparin as a ligand.

FIG. 4 is a graph showing an elution pattern of Glu-LBS-I in immunoaffinity chromatography using heparin as a ligand.

FIG. 5 shows results of SDS-PAGE (gel electrophoresis) analysis of the fractions of Lys-LBS-I with a high heparin binding activity.

FIG. 6 is a graph showing a heparin binding activity of Lys-LBS-I at various pHs.

FIG. 7 is a graph showing the effect of Lys-LBS-I in an inhibition test of lewis lung cancer metastasis and growth using C57BL6/J mice as compared to that of Glu-LBS-I.

FIG. 8 is a graph showing the effect of Lys-LBS-I in an inhibition test of lewis lung cancer metastasis and growth using SCID mice as compared to that of Glu-LBS-I.

FIG. 9 is a graph showing the activity to inhibit tumor metastasis and growth of the fractions of Lys-LBS-I with a high heparin binding activity as compared to that of the fractions of Lys-LDS-i with no heparin binding activity

### BEST MODE FOR CARRYING OUT THE INVENTION

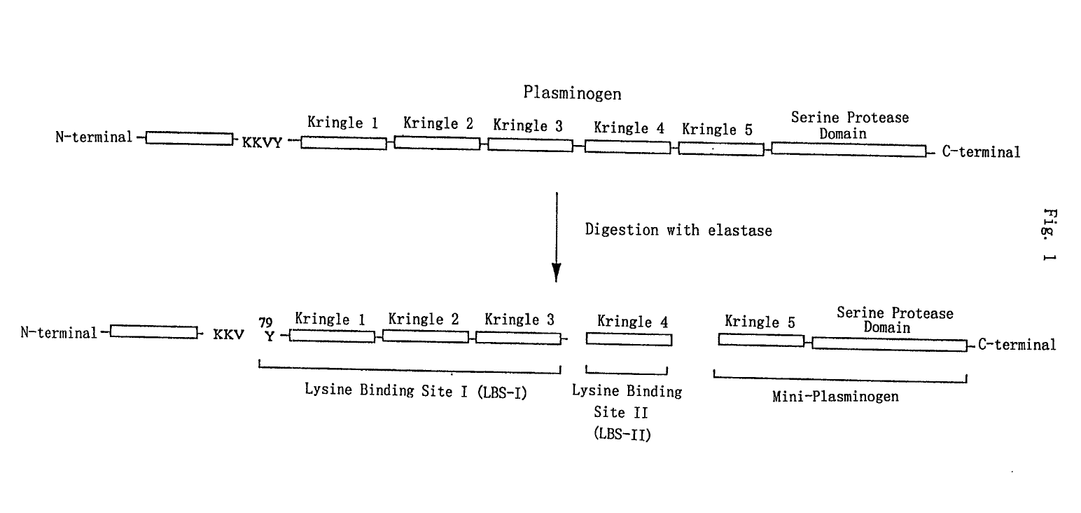

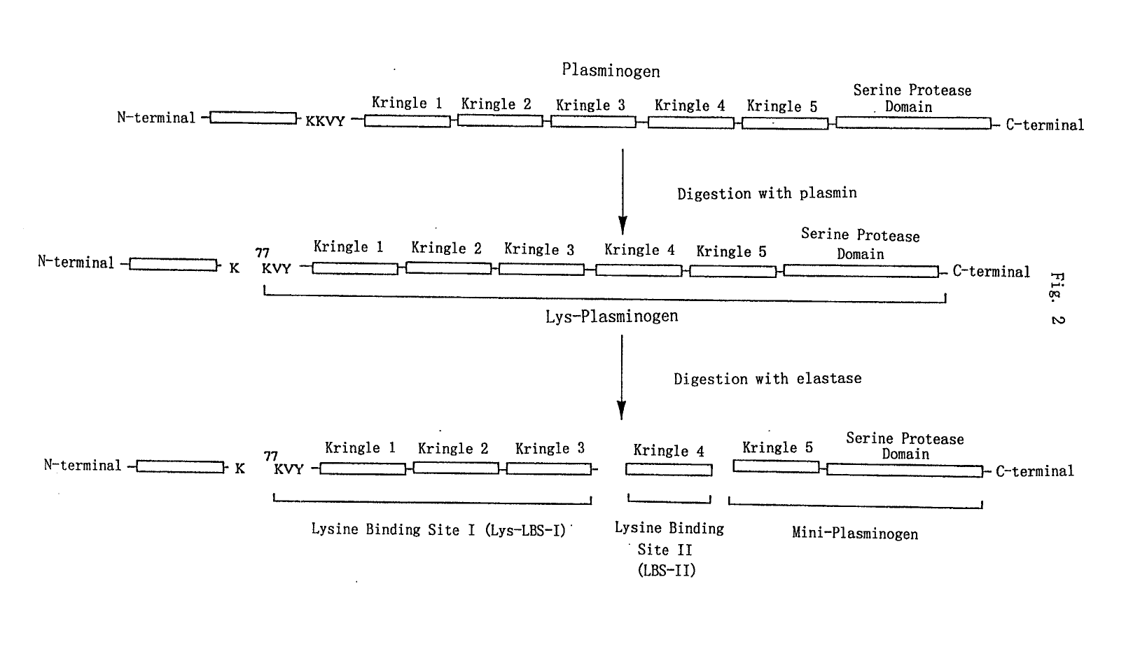

FIG. 1 depicts lysate products obtained by elastase digestion of plasminogen whereas FIG. 2 depicts lysate products obtained by plasmin digestion of plasminogen followed by elastase digestion in accordance with the present invention.

As shown in FIG. 1, when plasminogen is directly digested with elastase, restricted degradation of plasminogen occurs with cleavage at 78Val-79Tyr to release a fragment with N-terminal Tyr comprising Kringle 1 to Kringle 3 (Glu-LBS-I). In contrast, when plasminogen is first restrictedly digested with plasmin, it is restrictedly digested at 76Lys-77Lys to produce plasminogen with N-terminal Lys (Lys-Plg.), which is then cleaved with elastase to produce a fragment with N-terminal Lys comprising Kringle 1 to Kringle 3 (Lys-LBS-I) (FIG. 2). That is, with plasminogen with N-terminal Lys (Lys-Plg.), it is not cleaved at the 79Lys but instead LBS-I with N-terminal Lys is obtained. This is because conformation for elastase degradation might not be established at said N-terminal of Lys-Plg. In any way, it is of great interest to know that the activity to inhibit tumor metastasis and growth or the heparin binding activity may much vary depending on slight difference of only 1 or 2 amino acid residues at the N-terminal.

The plasminogen fragment with the activity to inhibit tumor metastasis and growth of the present invention is Lys-LBS-I obtained by elastase degradation of Lys-Plg. Among these fragments, those with a high heparin binding activity exhibit an especially strong activity to inhibit tumor metastasis and growth. Lys-LBS-I with N-terminal 77Lys comprising Kringle 1 to Kringle 3 is a protein with no glycosylation having a molecular weight of 38 Kda by SDS polyacrylamide gel electrophoresis (PAGE). This protein exhibits stronger heparin binding activity than other isotypes with glycosylation. Although said fragment cannot bind to heparin under physiological conditions, i.e. at around neutral pH at physiological ionic concentration, it can bind to heparin at physiological ionic concentration when the surrounding pH is inclined to be acidic.

Heparin has an activity to inhibit blood coagulation by binding to antithrombin III, one of plasmatic components. Heparin or heparin-like substance is widely distributed over the vascular endothelial cells aligned within the blood vessel to inhibit excess blood coagulation within the blood vessel. The plasminogen fragment of the present invention has the heparin binding activity. Thus, in addition to the conventional binding type used when plasminogen (plasmin) is bound to fibrin etc., such as binding to lysine, the plasminogen fragment of the present invention may possibly directly bind to the vascular endothelial cells to exert some activities.

For propagation of tumor cells at distal metastasized legion, a blood vessel is necessary for transporting nutrients to, and waste materials from, the tumor cells. Thus, in connection with propagation of tumor cells, a phenomenon that “a novel blood vessel is formed and guided to tumor cells (vascularization)” is observed. Vascularization occurs when the endothelial cells of the existing blood vessels, upon receipt of signal from the tumor cells, “propagate” and “elongate” towards the tumor cells and “vascularize” while destroying the existing blood vessels.

Thus, if the above behaviors of the endothelial cells are suppressed, then it might be possible to inhibit growth of tumor cells at distal metastasized legion. From this point of view, the heparin binding activity of the plasminogen fragment of the present invention is a requisite for exerting an inhibitory activity to the behaviors of the endothelial cells.

However, it should be noted that any blood vessels including the existing blood vessels have the endothelial cells being spread over the inside of blood vessel. Accordingly, an active substance must selectively be bound to only tumor cells so that it can suppress propagation of the tumor cells at distal metastasized legion alone without affecting to the endothelial cells of normal blood vessels. Jain et al. reported that blood vessels within the tumor legion are extremely polarized to result in stasis, and hence, environmental pH is inclined to be acidic due to hypoxic conditions [Jain R. K. et al., Barriers to drug delivery in solid tumors, Sci. Am. Vol. 271 (1) 58-65, 1994]. As explained above, the plasminogen fragment of the present invention does not bind to heparin or heparin-like substance under physiological (isotonic) conditions but attains the heparin binding activity under non-physiological conditions with a lower environmental pH. Thus, as reported by Jain et al., under acidic conditions such as within the tumor legion, the plasminogen fragment of the present invention binds to heparin or heparin-like substance in the tumor legion, and as a result, specifically affects to the tumor.

A mechanism wherein the plasminogen fragment of the present invention inhibits growth of the tumor cells remains unknown. However, viewing its structure, the inhibition mechanism might possibly be based on competitive inhibition of the plasmin activity.

As mentioned above, O'Reilly et al. reported that the plasminogen fragment directly inhibits growth of the endothelial cells of blood vessel. They found that cancer-bearing animals with grafted lewis lung cancer produced a substance having a strong angiogenesis inhibitory activity in blood and urine and purified said substance named Angiostatin. They reported that Angiostatin shows a high homology with internal fragments of plasminogen and purified human LBS-I (Glu-LBS-I) exhibits equivalent activity. The human LBS-I comprises three isotypes with a molecular weight ranging from 38 Kda to 42.5 Kda and is of great interest in view of its high homology with the plasminogen fragment of the present invention.

At present, it is not yet concluded whether the plasminogen fragment of the present invention and Angiostatin are the same substance with the same mechanism of action. However, there do exist difference between the plasminogen fragment of the present invention and Angiostatin in both their properties and biological activity. That is, (1) the plasminogen fragment of the present invention, not being associated with glycosylation, can inhibit growth of tumor cells at distal metastasized legion more significantly than other isotypes associated with glycosylation, and (2) the plasminogen fragment of the present invention less inhibits growth of the endothelial cells of blood vessel as compared to Angiostatin.

Thus, a possibility cannot be denied that the excellent activity to inhibit tumor metastasis and growth exerted by the plasminogen fragment of the present invention might possibly be due to the above difference from Angiostatin.

A process for preparing the plasminogen fragment of the present invention is not limited to a specific procedure. For example, it is produced by the following steps: (1) plasminogen is treated with plasmin etc. to produce Lys-Plg.; (2) a Lys-Plg.-containing solution is treated with elastase to give fractions of fragments (LysLBS-I) comprising from Kringle 1 to Kringle 3; and (3) among the obtained fractions, one having a strong heparin binding activity is selected to give a desired plasminogen fragment having the activity to inhibit tumor metastasis and growth.

Specifically, plasminogen is first isolated from blood samples and Lys-Plg. is prepared therefrom. A bloodderived plasminogen can be prepared as described below. For example, the method of Deutsch et al. using purification by affinity chromatography using a lysine carrier [Deutsch, D. G. et al., Science 170: 1095, 1970] and a modification of this method [Brockway, W. J. et al., Arch. Biochem. Biophys. 151: 194, 1972] may be typically employed. That is, plasminogen with high purity can be prepared by mixing fresh plasma with aprotinin (20 U/ml) and EDTA (2.5 mM), applying the mixture to a lysine carrier, washing the carrier with a buffer containing 0.1 M NaCl/2.5 mM EDTA/20 U aprotinin/ml and then with the same buffer supplemented with a surfactant, and eluting plasminogen with 6-aminohexanoic acid. Finally, purification and concentration is conducted with ultrafiltration membrane (e.g. YM10 manufactured by Amicon).

In blood, plasminogen is mostly present in the form of an intact molecule while Lys-Plg. with N-terminal Lys is merely found in a trace amount. Thus, in accordance with the present invention, such plasminogen in the form of an intact molecule must be transformed into Lys-Plg. This can be done by directly treating plasminogen with urokinase [Ljungberg, J. et al., Thromb. Res. 53: 569-576, 1989], by directly treating plasminogen with plasmin [Castellino, F. S. et al., Methods in Enzymology, Academic Press, New York, vol. 80, 365, 1981], or by incubating plasminogen for a long period of time [Markus, G. et al., J. Biol. Chem., vol. 254, 1211-1216, 1979], and the like. In a preferred embodiment of the present invention, plasminogen is incubated in the presence of tranexamic acid to autolysis to give Lys-Plg.

Then, the obtained Lys-Plg. is digested with elastase and, from the resulting fragments, a molecule comprising from Kringle 1 to Kringle 3 of Lys-Plg. (LysLBS-I) is collected by gel filtration using, for example, Sephadex G-75 and the following lysine-affinity chromatography to prepare successfully Lys-LBS-I. The obtained Lys-LBS-I is then contacted with a resin to which heparin is coupled as a ligand to thereby isolate bound fractions, from which those fractions strongly bound to heparin can specifically be prepared.

It is also possible to directly prepare the plasminogen fragment of the present invention, i.e. LysLBS-I, by means of the genetic engineering technique. That is, Lys-LBS-I can be prepared by constructing Lysplasminogen-producing cells by means of the genetic engineering technique and then digesting the resulting Lysplasminogen with elastase into fragments. Alternatively, a gene directly encoding the plasminogen fragment (Lys-LBS-I) of the present invention may be introduced into host cells such as eukaryotic cells, mammal cells or insect cells via an appropriate vector so that the desired plasminogen fragment is permanently produced.

The plasminogen fragment of the present invention prepared as above must be used while it is fresh or, when it is stored at 4° C., it is preferably used within about 5 days after storage. The plasminogen fragment of the present invention together with a stabilizing agent such as human albumin, gelatin, salt, sugar or amino acids can be lyophilized or stored in a liquid state. A solution of the plasminogen fragment of the present invention might also be frozen and stored. Furthermore, for inactivating infectious contaminant viruses, heat treatment is most preferably made to the lyophilizate or the liquid under suitable conditions, for example, at 65° C. for 96 hours for the lyophilizate or at 60° C. for 10 hours for the liquid, from viewpoint of safety of a drug.

The plasminogen fragment of the present invention may be formulated into an agent for inhibiting tumor metastasis and growth by combining said fragment as an active ingredient with suitable known excipients.

An effective dose of the agent for inhibiting tumor metastasis and growth comprising the plasminogen fragment of the present invention as an active ingredient may vary depending on various factors including, for example, age of subject, symptoms, and severity of the disease, etc. and will finally be left to the discretion of a physician. However, it may generally range from 50 to 500 mg/day for an adult, preferably 100 to 300 mg/day, which is administered in one to two portions. The agent is most preferably administered in a single bolus or instilled into the vein. The agent can also optionally be administered in combination with other anti-tumor agents. Thus, in one of preferred embodiments, the agent for inhibiting tumor metastasis and growth of the present invention may further comprise the other anti-tumor agents.

The present invention is illustrated in more detail hereinbelow by means of Examples but is not construed to be limited thereto. The blood-derived plasminogen fragment used in the following Examples is proved its safety by toxicity tests of single and multiple intravenous administration in mice, general pharmacological test to investigate effects on respiratory and circulatory systems using Beagles, virus inactivation test, and the like.

### Example 1

Preparation of plasminogen

To pooled fresh frozen plasma 10 L were added 20 mM benzamidine, 1 mM PMSF, and 100 U/ml aprotinin (Tradiol manufactured by Byer) and cold-thawed at room temperature. Then, the suspension was centrifuged with a high-speed centrifuge (RS-20IV manufactured by Tomy Seiko K. K.) at 8,000 rpm at 4° C. for 20 minutes to obtain supernatant. The supernatant was passed through Lysine-Sepharose 4B column (inner diameter 5.0×30 cm; manufactured by Pharmacia) equilibrated with 50 mM Tris/0.5 M NaCl (pH 7.5) at a flow rate of 1.0 ml/min. The column was washed with 5 volumes of the same buffer. The column was then eluted with the same buffer supplemented with 10 mM aminohexanoic acid. The eluate was dialyzed against 0.1 M ammonium carbonate buffer at 4° C. overnight.

### Example 2

Preparation of Lys-Plg.

After the eluate obtained by the chromatographic procedure in Example 1 was concentrated, it was dialyzed against 50 mM Tris/20 mM citrate buffer (pH 6.5) overnight. To the concentrate was added 1 mM tranexamic acid and the mixture was incubated at 30° C. overnight.

### Example 3

Preparation of elastase-Sepharose

Elastase type IV derived from porcine spleen (manufactured by Sigma) 50 mg was dissolved in a solution of 0.1 M sodium hydrogencarbonate containing 0.5 M NaCl and the solution was dialyzed against the same buffer at 4° C. overnight. CNBr-activated Sepharose 4 Fast Flow (manufactured by Pharmacia) was used for a gel for immobilizing elastase. Elastase was coupled to the gel in accordance with the manufacture's instruction at a ratio of elastase/gel of 5 mg/ml.

### Example 4

Preparation of elastase lysate of plasminogen and LysPlg.

Both plasminogens Glu-Plg. and Lys-Plg. prepared in Examples 1 and 2, respectively, were digested with elastase prepared in Example 3, as described in Davidson et al., (as above) to isolate elastase lysates of both plasminogens. Specifically, aprotinin 100 U/ml (Tradiol manufactured by Byer) was added to 10 mg/ml of the purified Glu-Plg. or Lys-Plg. and the mixture was dissolved in a solution of 0.1 M ammonium carbonate. Thereto was added the elastase-Sepharose at a ratio of enzyme/substrate 1:100 and the mixture was reacted at 25° C. overnight while stirring. After completion of the reaction, the reaction solution was filtered with a glass filter and the filtrate was passed through a lysine-Sepharose (manufactured by Pharmacia) equilibrated with 0.1 M and then the column was washed with the same buffer. Lysine-Sepharose binding fractions were eluted with the same buffer containing 20 mM aminohexanoic acid. The eluate was concentrated with ultrafiltration membrane YM-10 (manufactured by Amicon) and then passed through Sephadex G-75 column (inner diameter 5.0×40 cm; manufactured by Pharmacia) equilibrated with 0.1 M ammonium carbonate buffer to prepare Glu-Lysine Binding Site I (Glu-LBS-I) and Lys-Lysine Binding Site I (Lys-LBS-I), respectively. Both LBS-Is were lyophilized and stored at 4° C. till use.

### Example 5

Heparin binding activity

Glu-LBS-I and Lys-LBS-I prepared in Example 4 were passed through immunoaffinity chromatography Hi trap Heparin (trade name) (manufactured by Pharmacia) with heparin as a ligand. A concentration gradient elution based on a salt concentration was carried out to give heparin-binding fractions and proteins contained therein were monitored through absorbance for their heparin-affinity and amount.

Specifically, an immunoaffinity resin (1 ml) equilibrated with Tris buffer (pH 7.2) containing 50 mM NaCl was contacted with each 100 μl of Glu-LBS-I (1 mg/ml) and Lys-LBS-I (1 mg/ml) dissolved in the same buffer and washed with the same buffer 10 ml at a flow rate of 0.5 ml/min. Then, gradient elution was carried out with 50 mM NaCl/Tris 10 ml, 1 M NaCl/Tris buffer (pH 7.2) 10 ml.

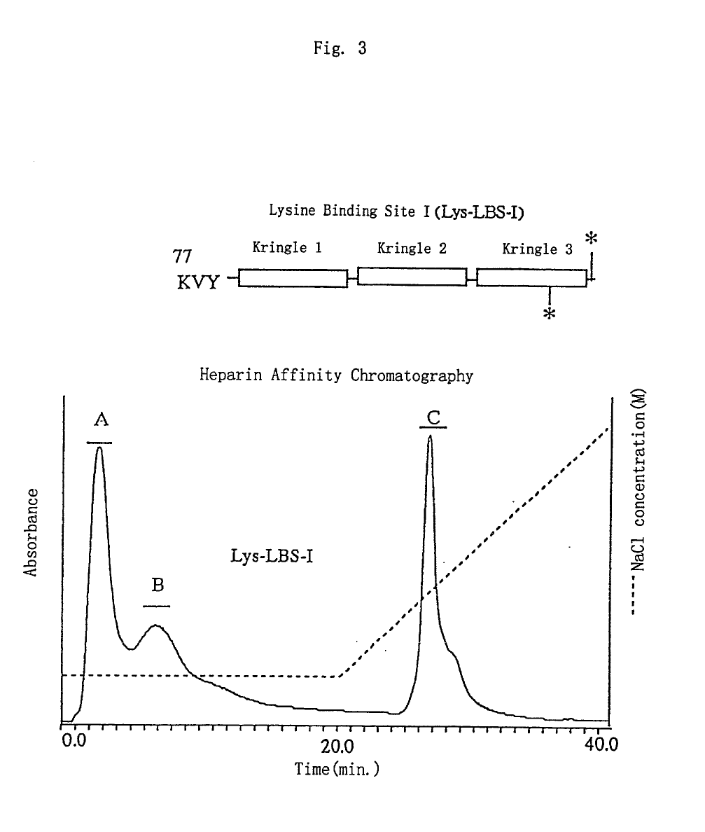

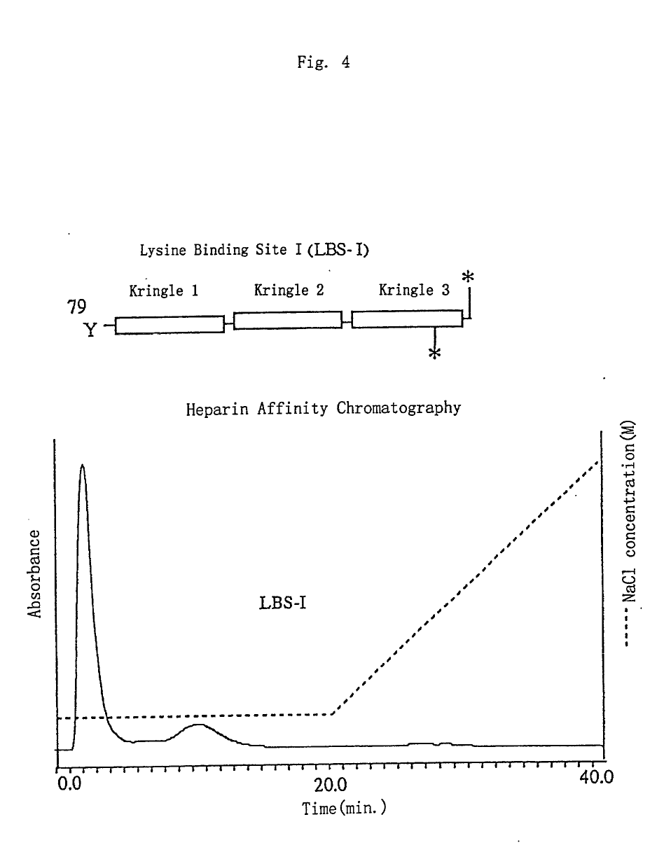

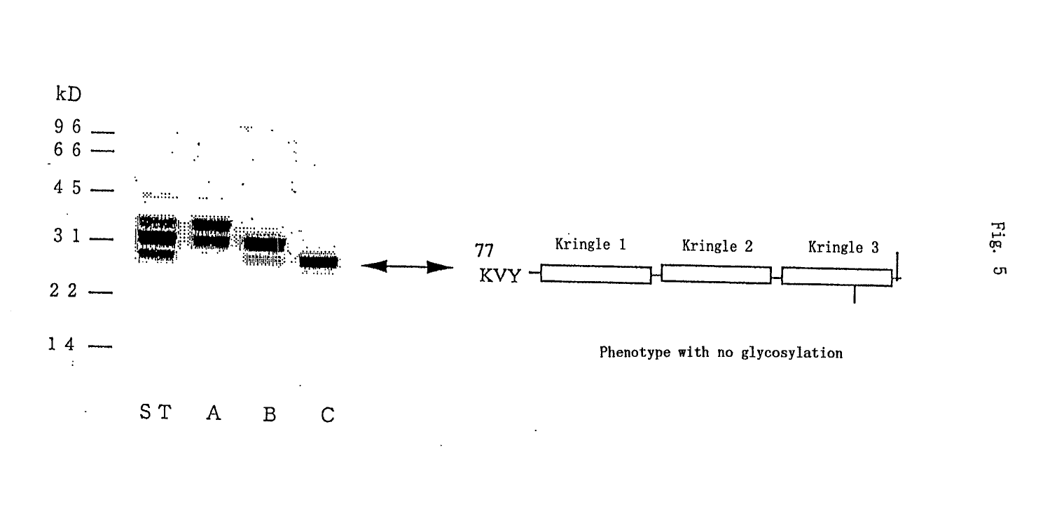

The results of heparin affinity chromatography are shown in FIGS. 3 and 4. As shown in FIG. 3, Lys-LBS-I gave fractions with no heparin binding activity, fractions with a moderate heparin binding activity and fractions with a high heparin binding activity whereas Glu-LBS-I gave no fractions with a high heparin binding activity (FIG. 4) The fractions with a high heparin binding activity were subjected to 12.5% SDS-PAGE to prove that the protein contained therein had a molecular weight of around 38 kda, which was consistent with that of LBS-I with no glycosylation (FIG. 5). The starting plasminogen did not dissolve in the above buffer for equilibrium and hence the immunoaffinity chromatography could not be done.

### Example 6

Relationship between heparin binding activity and pH

For Lys-LBS-I prepared in Example 4, immunoaffinity chromatography (Hi trap Heparin; manufactured by Pharmacia) with heparin as a ligand was carried out at a physiological saline at pH ranging from 5.0 to 7.2 to investigate the heparin binding activity. Specifically, the above immunoaffinity resin (1 ml) equilibrated with a citrate buffer (pH 5.0 to 7.2) containing 150 mM NaCl was contacted with 100 μl of LysLBS-I (1 mg/ml) dissolved in the same buffer and washed with the same buffer 10 ml at a flow rate of 0.5 ml/min, which was then eluted with 1 M NaCl/citrate buffer (pH 5.0 to 7.2) 10 ml.

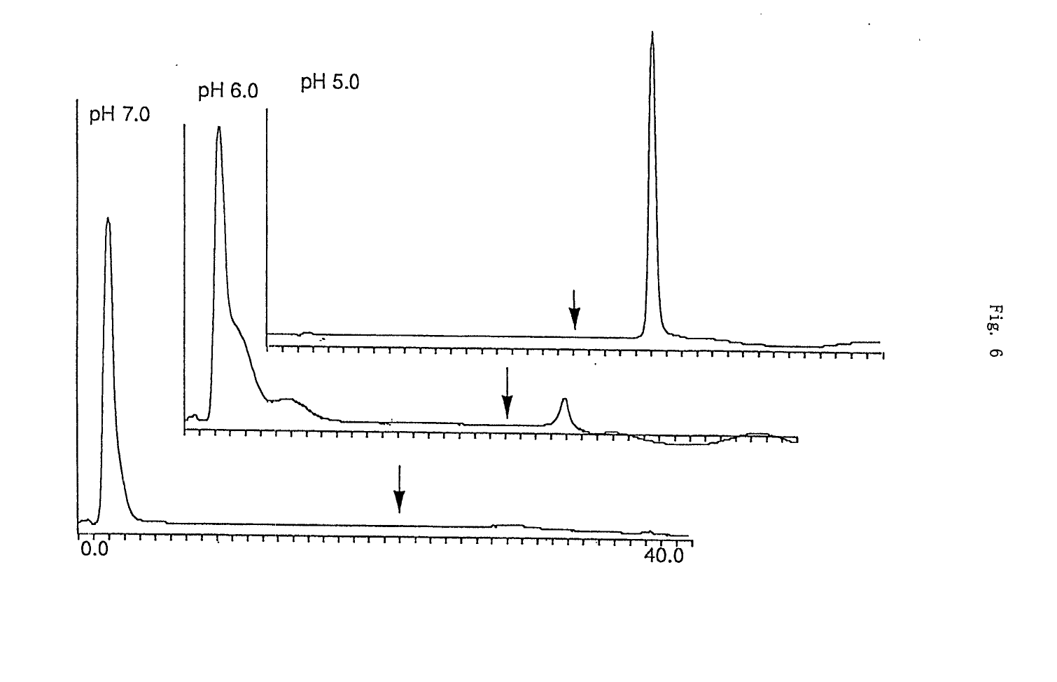

FIG. 6 shows a relationship between the heparin binding activity of Lys-LBS-I and pH. As shown in FIG. 6, Lys-LBS-I cannot bind to heparin under isotonic conditions at around neutral pH. However, with decrease in pH, the heparin binding activity increased wherein all the fractions bound to heparin at pH 5.0.

### Example 7

Preparation of human plasminogen fragment with heparin binding activity

Glu-LBS-I and Lys-LBS-I prepared in Example 4 were dialyzed against Tris buffer (pH 7.2) containing 50 mM NaCl overnight and then subjected to heparin affinity chromatography as described in Example 5 to prepare fractions with heparin binding activity at a concentration of 10 mg/ml. Hi trap Heparin 5 ml was contacted with each 3 ml of the above elastase-digested fragments 10 mg/ml and washed at a flow rate of 2.5 ml/min. for 40 minutes. Then, it was subjected to gradient elution with 1 M NaCl/Tris buffer (pH 7.2) 25 ml to prepare eluted fractions using a fraction collector (Readilack manufactured by Pharmacia). The fractions with the heparin binding activity were pooled and then dialyzed against 0.1 M ammonium carbonate buffer. The obtained dialyzate was subjected to sterile filtration and then lyophilized for use in animal tests.

### Example 8

Inhibition test of lung cancer metastasis and growth

For cancer cells, lewis lung cancer LL2 [Bertram, J. S. et al., Establishment of a cloned line of Lewis Lung Carcinoma cells adapted to cell culture: Cancer Lett. Vol. 11, 63-73, 1980] was purchased from Dainippon Seiyaku K. K. and cultured and subcultured in high concentration glucoseDMEM medium/10% FCS.

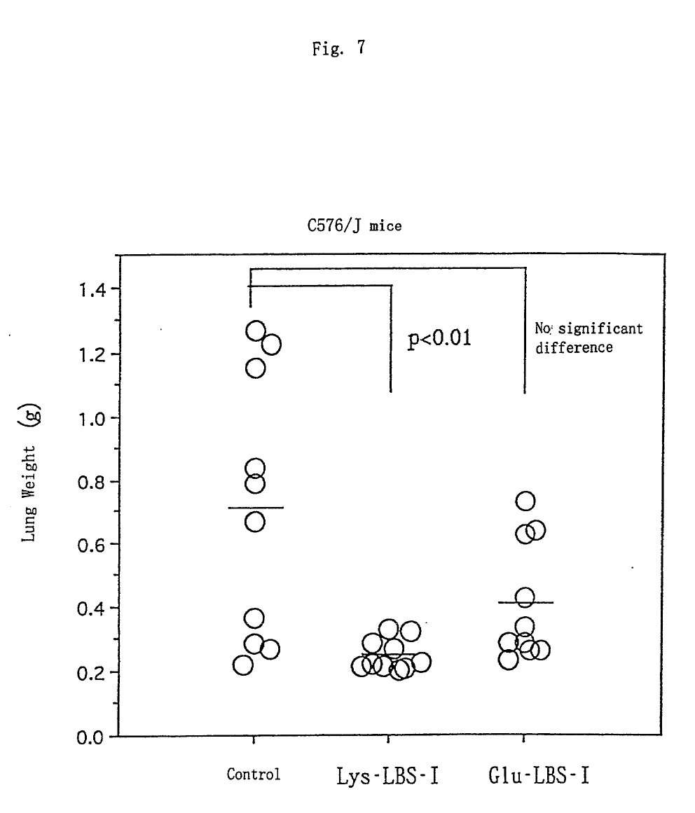

100 μl of Lewis lung cancer 10⁷ cells/ml was subcutaneously grafted to 30 male mice (C57BL6/J) of 6 weeks old at the back and the animals were bred for 15 to 18 days. Thereafter, the primary focus formed was surgically removed and the section was sutured. Taking body weight and weight of the primary focus into consideration, mice were dived into three groups and bred for 14 days. Mice in each group received intraperitoneal administration of each 0.5 mg/kg of either Lys-LBS-I or Glu-LBS-I prepared in Example 4 or 100 μl of physiological saline as a control everyday for 10 days. After administration, lungs were removed from mice and their weights were compared. The data were statistically analyzed using non-parametric analysis. FIG. 7 shows effects of Lys-LBS-I and Glu-LBS-I on tumor metastasis and growth. The lungs weighed 0.705±0.411 g in the control group (physiological saline) whereas they weighed 0.247±0.05 g in the Lys-LBS-I group to prove that Lys-LBS-I significantly inhibited tumor metastasis and growth. On the other hand, the lungs weighed 0.406±0.186 g in the Glu-LBS-I group with no significant difference.

### Example 9

Inhibition test of lung cancer metastasis and growth using immunologically deficient animals

The experiment in Example 8 was repeated except that mice were replaced with immunologically deficient animals, SCID mice, to investigate effects on lung cancer metastasis and growth.

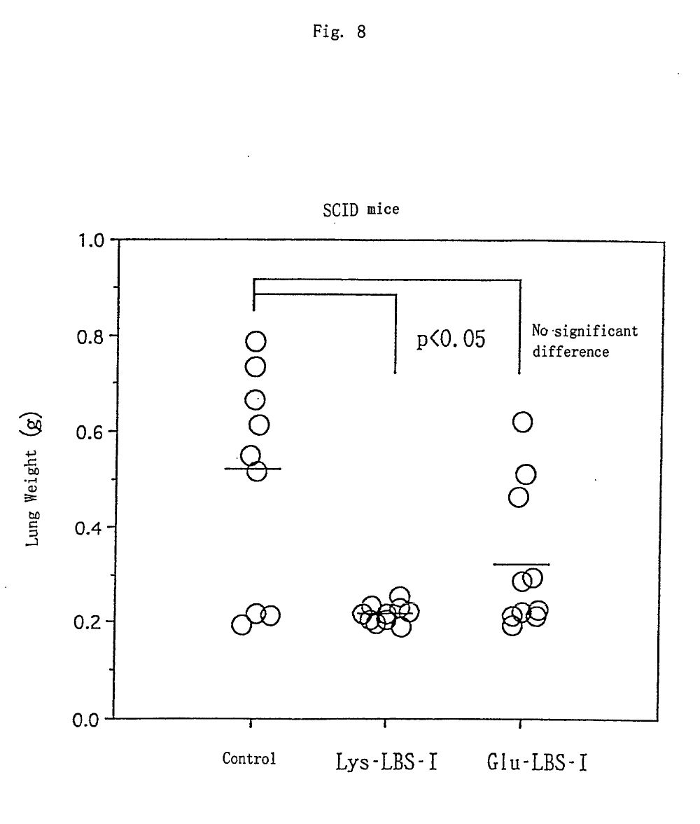

FIG. 8 shows results obtained in this test model in which immunological effects due to continuous administration of heterologous proteins were taken into consideration. The lungs weighed 0.522±0.232 g, 0.217±0.019 g and 0.324±0.152 g in the physiological saline group (control group), the Lys-LBS-I group and the Glu-LBSI group, respectively, which are similar results as in

### Example 8.

### Example 10

Inhibitory effects of fractions with heparin binding activity on tumor metastasis and growth

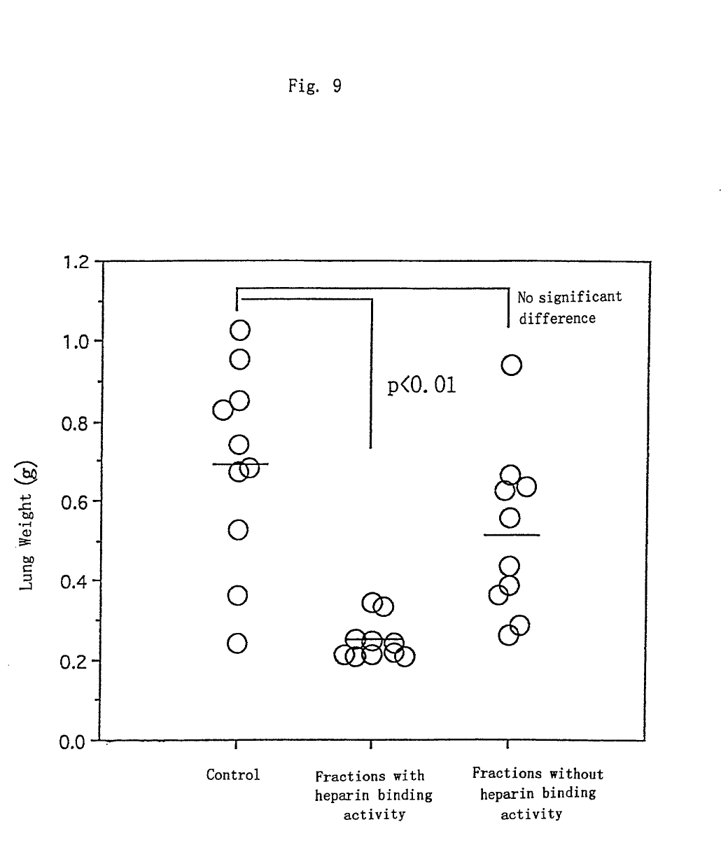

Inhibitory effects of the fractions with and without heparin binding activity prepared in Example 7 on tumor metastasis and growth were investigated as described in Example 8. The results are shown in FIG. 9. The lungs weighed 0.689±0.250 g in the control group (physiological saline) whereas they weighed 0.248±0.05 g in the group of the fractions with heparin binding activity to prove that the fractions with heparin binding activity significantly inhibited tumor metastasis and growth. On the other hand, the lungs weighed 0.515±0.208 g in the group of the fractions without heparin binding activity with no significant difference.

### Example 11

Determination of N-terminal amino acid sequence of fragments with heparin binding activity

The fractions with heparin binding activity prepared in Example 7 were electrophoresed on 12.5% SDS-PAGE and transferred to a membrane in the conventional manner. The obtained bands were excised and the N-terminal amino acid residue of the proteins contained in said bands was determined using N-terminal amino acid sequence analyzer (manufactured by Bio Applied). As a result, it was found that the bands contained a mixture of two proteins with the N-terminal amino acid of either Lys or Val at a ratio of 1.56 to 2.3:1.

1. A Lys-lys binding site I which is a plasminogen fragment consisting of Kringle 1 to Kringle 3 of a naturally occurring plasminogen with the N-terminal being lysine, which binding site binds to heparin and has the following properties:
a. a molecular weight of 38 kDa;
b. it is not glycosylated;
c. it binds intensely to heparin under nonphysiological conditions but binds less intensely to heparin under physiological conditions;
d. it inhibits tumor metastasis and tumor growth but has no ability to inhibit growth of endothelial cells of blood vessels;
wherein said plasminogen fragment is prepared by:
a. preparing Lys-plasminogen from naturally occurring plasminogen either by adding plasmin to a solution of naturally occurring plasminogen or by incubating naturally occurring plasminogen in the presence of tranexamic acid to autolysis;
b. treating the Lys-plasminogen obtained in step (a) with elastase to produce fractions of the fragment comprising Kringle 1 to Kringle 3;
c. identifying the fragment of Kringle 1 to Kringle 3 which binds to heparin.

2. A process for preparing a plasminogen fragment consisting of Kringle 1 to Kringle 3 of a naturally occurring plasminogen with the N-terminal being lysine, said fragment having the ability to inhibit tumor metastasis and tumor growth, but having no ability to inhibit growth of endothelial cells of blood vessels, comprising:
a. preparing Lys-plasminogen from naturally occurring plasminogen either by adding plasmin to a solution of naturally occurring plasminogen or by incubating naturally occurring plasminogen in the presence of tranexamic acid to autolysis;
b. treating the Lys-plasminogen obtained in step (a) with elastase to produce fractions of the fragment consisting of Kringle 1 to Kringle 3;
c. identifying the fragment of Kringle 1 to Kringle 3 which binds to heparin; and
d. isolating the fragment which binds to heparin.

3. The process according to claim 2 wherein the fragment which bind to heparin is recovered by passing a solution of a Lys-plasminogen lysate with elastase through a carrier to which heparin is coupled as a ligand to adsorb those fragment which bind to heparin, and eluting those fragments which do not bind to heparin.

4. A composition for inhibiting tumor metastasis and tumor growth comprising an effective amount of a fragment according to claim 1 and, optionally, a pharmaceutically acceptable carrier.
